SEQUENCE LISTING (14 sequences)

SEQ ID NO: 1 - DNA, 2814 bp, Mus musculus
tttgttgtcattggcggctcccaagatggcgtccatcgtggaagggccgctgagcaaatggactaacgtgatgaagggatggcagtatcgttggttcgtgctggactacaatgcagggctgctctcctactacacgtccaaggacaaaatgatgagaggctctcgaagaggatgcgttagactcagaggagctgtgattggtatagacgacgaggacgacagcaccttcacaatcactgtcgatcagaaaaccttccacttccaggctcgagatgcagacgagcgagagaagtggatccatgccttagaagaaactattcttcgccatactcttcagcttcaaggtttggattcaggattcatccccagtgtccaagactttgataagaaacttaccgaggctgacgcgtacctgcagatcttgatagaacaattaaagctttttgatgacaagcttcaaaattgtaaagatgatgaacagagaaagaaagttgaaactctcaaagacacaacaaatagcatggtagaatcaattaaacactgcattgtgttgctacagattgctaaaagtactattaatcctgtagatgcaatataccagcctagtcccttggaacctgtgatcagcacaatgccttcccagactgccttacctccagaacccgctcagttgtgtaagtcagagcagcgtccatcttccttacctgttggacctgtgttagctaccttgggacatcatcagactccaacaccaaatagtacaggcagtgggaactcaccacctagcagcagtctgactcctcccagccatgtcaacttgtctccaaatacagtcccagagttctcttactctagcagtgaagatgagttctatgatgctgatgaattccatcaaagtggctcgtccccaaagcgcttaatagattcttctggatctgcctcagtcttgacacacagcagctccggaaatagcttaaaacgcccagataccacagagtctctgaattcctccatgtccaatggcacaagcgatgctgatctttttgactcacatgacgacagagatgatgatggggaggctgggtcagtggaggagcacaagagcgttatcatgcacctcttatcacaagtcaggctggggatggacctcacaaaggtagttcttccaacgtttattctcgagagaagatctctgttagaaatgtatgcagactttttcgcacatccagacctgttcgtgagcattagtgatcagaaggatcccagggatcgaatggttcaggttgtgaaatggtacctctcggccttccatgcaggaaggagaggatcggtggccaaaaagccgtacaatcctattttgggtgagatctttcagtgtcactggacgttgccgaatgatactgaagagaacgcagagctcgtttcagaagggccggttccctgggtttctaagaacagtgtaacatttgtggctgagcaagtttcccaccatccgcccatttcagccttttatgctgagtgttttaacaagaagatacaattcaatgctcatatctggactaaatcaaaattccttgggatgtcaattggggtacacaacataggtcagggctgtgtctcgtgtctggagtacgatgagcactacatcctcacgttccccaatggctatggaaggtctatcctgacagtgccctgggtggaattgggaggagaatgcaatatcaactgctccaaaacgggttacagcgcaaacatcgtcttccacactaagcctttctatgggggcaagaagcacagaattactgcagagattttttctccgaatgacaagaaatccttctgctcaattgaaggggaatggaatggtatcatgtatgcaaaatacgcaacaggggaaaacactgtctttgtagacaccaagaaattgcctataatcaagaaaaaggtgaggaagttggaagatcagaatgagtatgagtcccgcagcctttggaaggatgtcactttcaatttaaaaatcagagacattgatgcagcaacggaagcaaagcacagacttgaagaaagacaaagagcagaagcccgagaaaggaaggagaaggaaattcagtgggagacgaggctctttcacgaggatggcgaatgctgggtttacgatgagcctttactgaagcgtcttggtgctgtgaagcattaggccgacaaccgagtccacacctggtgaccagggcagtaggcgtaattaagcaacaatcgatcttccttcaggagagcttgtcacttccttcttaacgcagtggttcctatctcagggatactggacttgacgacacagatgaacaattaaagtggaaaccgcttcccttttctcctctgtggcagttacaattttgacttcaggcctgagaaaaacttcaggtttcgaaacatgacatctcttccttttccagatcccatgctttgaaaaatatttatagacagttccaggtctcagcttcctgtcctctagttctgctgttcgggcataaaatctttatctccagttcatataatcttgagttttagatakacacacatgcgtaacagctgacagtttttcacaagtacacccacctgtaaatactgtakcctcagtttagaaaattagtgcatgtatgaaaatcaagtgttaggaaatttcatggtttcacctataacctttattttagaattgaactatgattagattgtatctaaacctgaagtataattatatgcagtgcttcttaaggcttcataaaataattttccaacctttttaaaaaaaaaaaaaaa

SEQ ID NO: 2 - protein, 723 aa, Mus musculus
MASIVEGPLSKWTNVMKGWQYRWFVLDYNAGLLSYYTSKDKMMRGSRRGCVRLRGAVIGIDDEDDSTFTITVDQKTFHFQARDADEREKWIHALEETILRHTLQLQGLDSGFIPSVQDFDKKLTEADAYLQILIEQLKLFDDKLQNCKDDEQRKKVETLKDTTNSMVESIKHCIVLLQIAKSTINPVDAIYQPSPLEPVISTMPSQTALPPEPAQLCKSEQRPSSLPVGPVLATLGHHQTPTPNSTGSGNSPPSSSLTPPSHVNLSPNTVPEFSYSSSEDEFYDADEFHQSGSSPKRLIDSSGSASVLTHSSSGNSLKRPDTTESLNSSMSNGTSDADLFDSHDDRDDDGEAGSVEEHKSVIMHLLSQVRLGMDLTKVVLPTFILERRSLLEMYADFFAHPDLFVSISDQKDPRDRMVQVVKWYLSAFHAGRRGSVAKKPYNPILGEIFQCHWTLPNDTEENAELVSEGPVPWVSKNSVTFVAEQVSHHPPISAFYAECFNKKIQFNAHIWTKSKFLGMSIGVHNIGQGCVSCLEYDEHYILTFPNGYGRSILTVPWVELGGECNINCSKTGYSANIVFHTKPFYGGKKHRITAEIFSPNDKKSFCSIEGEWNGIMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAVKH

SEQ ID NO: 3 - DNA, 2534 bp, Mus musculus
gggcccctcccctgagacggcggccggccgccgcgaatggctctcctgctggccgcttgcggaggtttggattcaggattcatccccagtgtccaagactttgataagaaacttaccgaggctgacgcgtacctgcagatcttgatagaacaattaaagctttttgatgacaagcttcaaaattgtaaagatgatgaacagagaaagaaagttgaaactctcaaagacacaacaaatagcatggtagaatcaattaaacactgcattgtgttgctacagattgctaaaagtactattaatcctgtagatgcaatataccagcctagtcccttggaacctgtgatcagcacaatgccttcccagactgccttacctccagaacccgctcagttgtgtaagtcagagcagcgtccatcttccttacctgttggacctgtgttagctaccttgggacatcatcagactccaacaccaaatagtacaggcagtgggaactcaccacctagcagcagtctgactcctcccagccatgtcaacttgtctccaaatacagtcccagagttctcttactctagcagtgaagatgagttctatgatgctgatgaattccatcaaagtggctcgtccccaaagcgcttaatagattcttctggatctgcctcagtcttgacacacagcagctccggaaatagcttaaaacgcccagataccacagagtctctgaattcctccatgtccaatggcacaagcgatgctgatctttttgactcacatgacgacagagatgatgatggggaggctgggtcagtggaggagcacaagagcgttatcatgcacctcttatcacaagtcaggctggggatggacctcacaaaggtagttcttccaacgtttattctcgagagaagatctctgttagaaatgtatgcagactttttcgcacatccagacctgttcgtgagcattagtgatcagaaggatcccagggatcgaatggttcaggttgtgaaatggtacctctcggccttccatgcaggaaggagaggatcggtggccaaaaagccgtacaatcctattttgggtgagatctttcagtgtcactggacgttgccgaatgatactgaagagaacgcagagctcgtttcagaagggccggttccctgggtttctaagaacagtgtaacatttgtggctgagcaagtttcccaccatccgcccatttcagccttttatgctgagtgttttaacaagaagatacaattcaatgctcatatctggactaaatcaaaattccttgggatgtcaattggggtacacaacataggtcagggctgtgtctcgtgtctggagtacgatgagcactacatcctcacgttccccaatggctatggaaggtctatcctgacagtgccctgggtggaattgggaggagaatgcaatatcaactgctccaaaacgggttacagcgcaaacatcgtcttccacactaagcctttctatgggggcaagaagcacagaattactgcagagattttttctccgaatgacaagaaatccttctgctcaattgaaggggaatggaatggtatcatgtatgcaaaatacgcaacaggggaaaacactgtctttgtagacaccaagaaattgcctataatcaagaaaaaggtgaggaagttggaagatcagaatgagtatgagtcccgcagcctttggaaggatgtcactttcaatttaaaaatcagagacattgatgcagcaacggaagcaaagcacagacttgaagaaagacaaagagcagaagcccgagaaaggaaggagaaggaaattcagtgggagacgaggctctttcacgaggatggcgaatgctgggtttacgatgagcctttactgaagcgtcttggtgctgtgaagcattaggccgacaaccgagtccacacctggtgaccagggcagtaggcgtaattaagcaacaatcgatcttccttcaggagagcttgtcacttccttcttaacgcagtggttcctatctcagggatactggacttgacgacacagatgaacaattaaagtggaaaccgcttcccttttctcctctgtggcagttacaattttgacttcaggcctgagaaaaacttcaggtttcgaaacatgacatctcttccttttccagatcccatgctttgaaaaatatttatagacagttccaggtctcagcttcctgtcctctagttctgctgttcgggcataaaatctttatctccagttcatataatcttgagttttagatakacacacatgcgtaacagctgacagtttttcacaagtacacccacctgtaaatactgtakcctcagtttagaaaattagtgcatgtatgaaaatcaagtgttaggaaatttcatggtttcacctataacctttattttagaattgaactatgattagattgtatctaaacctgaagtataattatatgcagtgcttcttaaggcttcataaaataattttccaacctttttaaaaaaaaaaaaaaa

SEQ ID NO: 4 - protein, 626 aa, Mus musculus
MALLLAACGGLDSGFIPSVQDFDKKLTEADAYLQILIEQLKLFDDKLQNCKDDEQRKKVETLKDTTNSMVESIKHCIVLLQIAKSTINPVDAIYQPSPLEPVISTMPSQTALPPEPAQLCKSEQRPSSLPVGPVLATLGHHQTPTPNSTGSGNSPPSSSLTPPSHVNLSPNTVPEFSYSSSEDEFYDADEFHQSGSSPKRLIDSSGSASVLTHSSSGNSLKRPDTTESLNSSMSNGTSDADLFDSHDDRDDDGEAGSVEEHKSVIMHLLSQVRLGMDLTKVVLPTFILERRSLLEMYADFFAHPDLFVSISDQKDPRDRMVQVVKWYLSAFHAGRRGSVAKKPYNPILGEIFQCHWTLPNDTEENAELVSEGPVPWVSKNSVTFVAEQVSHHPPISAFYAECFNKKIQFNAHIWTKSKFLGMSIGVHNIGQGCVSCLEYDEHYILTFPNGYGRSILTVPWVELGGECNINCSKTGYSANIVFHTKPFYGGKKHRITAEIFSPNDKKSFCSIEGEWNGIMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAVKH

SEQ ID NO: 5 - DNA, 2840 bp, Homo sapiens
tcccaagatggcgtccatcatggaagggccgctgagcaaatggactaacgtgatgaagggctggcagtaccgttggttcgtgctggactacaatgcaggactgctctcctactacacgtccaaggacaaaatgatgagaggctctcgcagaggatgtgttagactcagaggagctgtgattggtatagacgatgaggacgacagcaccttcacaataactgttgatcagaaaaccttccatttccaggcccgtgatgctgatgagcgagagaagtggatccatgccttagaagaaacaattcttcgacatactctccagcttcaaggtttggattcaggatttgttcctagtgtccaagattttgataagaaacttacagaagctgatgcttacctacaaatcttgattgaacaattaaagctttttgatgacaagcttcaaaactgcaaagaagatgaacagagaaagaaaattgaaactctcaaagagacaacaaatagcatggtagaatcaattaaacactgcattgtgttgctgcagattgccaaaagtactattaatcccgtagatgcaatatatcaacctagtcctttggaacctgtgatcagcacaatgccttcccagactgtgttacctccagaacctgttcagttgtgtaagtcagagcagcgtccatcttccctaccagttggacctgtgttggctaccttgggacatcatcagactcctacaccaaatagtacaggcagtggccattcaccaccgagtagcagtctcacttctccaagccacgtgaacttgtctccaaatacagtcccagagttctcttactccagcagtgaagatgaattttatgatgctgatgaattccatcaaagtggctcatccccaaagcgcttaatagattcttctggatctgcctcagtcctgacacacagcagctcgggaaatagtctaaaacgcccagataccacagaatcacttaattcttccttgtccaatggaacaagtgatgctgacctgtttgattcacatgatgacagagatgatgatgcggaggcagggtctgtggaggagcacaagagcgttatcatgcatctcttgtcgcaggttagacttggaatggatcttactaaggtagttcttccaacgtttattcttgaaagaagatctcttttagaaatgtatgcagacttttttgcacatccggacctgtttgtgagcattagtgaccagaaggatcccaaggatcgaatggttcaggttgtgaaatggtacctctcagcctttcatgcgggaaggaaaggatcagttgccaaaaagccatacaatcccattttgggcgagatttttcagtgtcattggacattaccaaatgatactgaagagaacacagaactagtttcagaaggaccagttccctgggtttccaaaaacagtgtaacatttgtggctgagcaggtttcccatcatccacccatttcagccttttatgctgagtgttttaacaagaagatacaattcaatgctcatatctggaccaaatcaaaattccttgggatgtcaattggggtgcacaacatagggcagggctgtgtctcatgtctagactatgatgaacattacattctcacattccccaatggctatggaaggtctatcctcacagtgccctgggtggaattaggaggagaatgcaatattaattgttccaaaacaggctatagtgcaaatatcatcttccacactaaacccttctatgggggcaagaagcacagaattactgccgagattttttctccaaatgacaagaagtctttttgctcaattgaaggggaatggaatggtgtgatgtatgcaaaatatgcaacaggggaaaatacagtctttgtagataccaagaagttgcctataatcaagaagaaagtgaggaagttggaagatcagaacgagtatgaatcccgcagcctttggaaggatgtcactttcaacttaaaaatcagagacattgatgcagcaactgaagcaaagcacaggcttgaagaaagacaaagagcagaagcccgagaaaggaaggagaaggaaattcagtgggagacaaggttatttcatgaagatggagaatgctgggtttatgatgaaccattactgaaacgtcttggtgctgccaagcattaggttggaagatgcaaagtttatacctgatgatcagggcagtaggcataattcagcaacaaacaatcttcctttgggagaaacctgttcattccaatcttctaattacagtggttcctatctcagggatactggactttctgacgcagatgaacaattaaggggaaaagcttcccttttccctctgtggcagttacgattttgacttcagtcctgagaaaaacttcaggttttgaaaatcagatgatgtcttctccttttccaaacaccacacgttgaaagcatttataaatccaagtctgaaactctgcgctctagtactgctgttaagatacacaacttgtttcttagttcatataatctcgggatacacacacacacacatatatatatacacacacatacgtatacacacacatacatatataaatatacctgatgccagatttttttcataaatattctgcctactgtaaatatgggttcctctgagttgttttagaaaattagcgcaatgtattaaaatcaagtgttaggaaatttcatggtcttacctacaataacttttattttggaattgaactattattaaattgtatctaatcctggattacagtttaattaattattcttagtgcttaaggcttcataaagtaatttttcc

SEQ ID NO: 6 - protein, 723 aa, Homo sapiens
MASIMEGPLSKWTNVMKGWQYRWFVLDYNAGLLSYYTSKDKMMRGSRRGCVRLRGAVIGIDDEDDSTFTITVDQKTFHFQARDADEREKWIHALEETILRHTLQLQGLDSGFVPSVQDFDKKLTEADAYLQILIEQLKLFDDKLQNCKEDEQRKKIETLKETTNSMVESIKHCIVLLQIAKSTINPVDAIYQPSPLEPVISTMPSQTVLPPEPVQLCKSEQRPSSLPVGPVLATLGHHQTPTPNSTGSGHSPPSSSLTSPSHVNLSPNTVPEFSYSSSEDEFYDADEFHQSGSSPKRLIDSSGSASVLTHSSSGNSLKRPDTTESLNSSLSNGTSDADLFDSHDDRDDDAEAGSVEEHKSVIMHLLSQVRLGMDLTKVVLPTFILERRSLLEMYADFFAHPDLFVSISDQKDPKDRMVQVVKWYLSAFHAGRKGSVAKKPYNPILGEIFQCHWTLPNDTEENTELVSEGPVPWVSKNSVTFVAEQVSHHPPISAFYAECFNKKIQFNAHIWTKSKFLGMSIGVHNIGQGCVSCLDYDEHYILTFPNGYGRSILTVPWVELGGECNINCSKTGYSANIIFHTKPFYGGKKHRITAEIFSPNDKKSFCSIEGEWNGVMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAAKH

SEQ ID NO: 7 - DNA, 2457 bp, Homo sapiens
cggcccctacccctgagtccccggggtcccggccgccaggccggagcgcgaatgtcgtgctcaccctgcctccttcccgccgccccctgggctttttgatgacaagcttcaaaactgcaaagaagatgaacagagaaagaaaattgaaactctcaaagagacaacaaatagcatggtagaatcaattaaacactgcattgtgttgctgcagattgccaaaagtactattaatcccgtagatgcaatatatcaacctagtcctttggaacctgtgatcagcacaatgccttcccagactgtgttacctccagaacctgttcagttgtgtaagtcagagcagcgtccatcttccctaccagttggacctgtgttggctaccttgggacatcatcagactcctacaccaaatagtacaggcagtggccattcaccaccgagtagcagtctcacttctccaagccacgtgaacttgtctccaaatacagtcccagagttctcttactccagcagtgaagatgaattttatgatgctgatgaattccatcaaagtggctcatccccaaagcgcttaatagattcttctggatctgcctcagtcctgacacacagcagctcgggaaatagtctaaaacgcccagataccacagaatcacttaattcttccttgtccaatggaacaagtgatgctgacctgtttgattcacatgatgacagagatgatgatgcggaggcagggtctgtggaggagcacaagagcgttatcatgcatctcttgtcgcaggttagacttggaatggatcttactaaggtagttcttccaacgtttattcttgaaagaagatctcttttagaaatgtatgcagacttttttgcacatccggacctgtttgtgagcattagtgaccagaaggatcccaaggatcgaatggttcaggttgtgaaatggtacctctcagcctttcatgcgggaaggaaaggatcagttgccaaaaagccatacaatcccattttgggcgagatttttcagtgtcattggacattaccaaatgatactgaagagaacacagaactagtttcagaaggaccagttccctgggtttccaaaaacagtgtaacatttgtggctgagcaggtttcccatcatccacccatttcagccttttatgctgagtgttttaacaagaagatacaattcaatgctcatatctggaccaaatcaaaattccttgggatgtcaattggggtgcacaacatagggcagggctgtgtctcatgtctagactatgatgaacattacattctcacattccccaatggctatggaaggtctatcctcacagtgccctgggtggaattaggaggagaatgcaatattaattgttccaaaacaggctacagtgcaaatatcatcttccacactaaacccttctatgggggcaagaagcacagaattactgccgagattttttctccaaatgacaagaagtctttttgctcaattgaaggggaatggaatggtgtgatgtatgcaaaatatgcaacaggggaaaatacagtctttgtagataccaagaagttgcctataatcaagaagaaagtgaggaagttggaagatcagaacgagtatgaatcccgcagcctttggaaggatgtcactttcaacttaaaaatcagagacattgatgcagcaactgaagcaaagcacaggcttgaagaaagacaaagagcagaagcccgagaaaggaaggagaaggaaattcagtgggagacaaggttatttcatgaagatggagaatgctgggtttatgatgaaccattactgaaacgtcttggtgctgccaagcattaggttggaagatgcaaagtttatacctgatgatcagggcagtaggcataattcagcaacaaacaatcttcctttgggagaaacctgttcattccaatcttctaattacagtggttcctatctcagggatactggactttctgacgcagatgaacaattaaggggaaaagcttcccttttccctctgtggcagttacgattttgacttcagtcctgagaaaaacttcaggttttgaaaatcagatgatgtcttctccttttccaaacaccacacgttgaaagcatttataaatccaagtctgaaactctgcgctctagtactgctgttaagatacacaacttgtttcttagttcatataatctcgggatacacacacacacacacatatatatacacacacatacgtatacacacacatacatatatataaatatacctgatgccagatttttttcataaatattctgcctactgtaaatatgggttcctctgagttgttttagaaaattagcgcaatgtattaaaatcaagtgttaggaaatttcatggtcttacctacaataacttttattttggaattgaactattattaaattgtatctaatcctgg

SEQ ID NO: 8 - protein, 558 aa, Homo sapiens
MVESIKHCIVLLQIAKSTINPVDAIYQPSPLEPVISTMPSQTVLPPEPVQLCKSEQRPSSLPVGPVLATLGHHQTPTPNSTGSGHSPPSSSLTSPSHVNLSPNTVPEFSYSSSEDEFYDADEFHQSGSSPKRLIDSSGSASVLTHSSSGNSLKRPDTTESLNSSLSNGTSDADLFDSHDDRDDDAEAGSVEEHKSVIMHLLSQVRLGMDLTKVVLPTFILERRSLLEMYADFFAHPDLFVSISDQKDPKDRMVQVVKWYLSAFHAGRKGSVAKKPYNPILGEIFQCHWTLPNDTEENTELVSEGPVPWVSKNSVTFVAEQVSHHPPISAFYAECFNKKIQFNAHIWTKSKFLGMSIGVHNIGQGCVSCLDYDEHYILTFPNGYGRSILTVPWVELGGECNINCSKTGYSANIIFHTKPFYGGKKHRITAEIFSPNDKKSFCSIEGEWNGVMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAAKH

SEQ ID NO: 9 - DNA, 21 bp, Artificial
  Synthetic
ccaagctctaatacgactcac

SEQ ID NO: 10 - DNA, 20 bp, Artificial
  Synthetic
ctcagccacaaatgttacac

SEQ ID NO: 11 - DNA, 32 bp, Artificial
  Synthetic
ctagctagcaccatggcgtccatcgtggaagg

SEQ ID NO: 12 - DNA, 35 bp, Artificial
  Synthetic
ataagaatgcggccgcgaggagaaaagggaagcgg

SEQ ID NO: 13 - DNA, 35 bp, Artificial
  Synthetic
ctagctagcaccatggctctcctgctggccgcttg

SEQ ID NO: 14 - DNA, 29 bp, Artificial
  Synthetic
cccgggcatgcttcacagcaccaagacgc

(19) United States
(12) Patent Application Publication
(10) Pub. No.: US 2006/0035314 A1
(43) Pub. Date: Feb. 16, 2006

(54) Receptors and signalling proteins capable of binding meiotic acting sterols (MAS)

(76) Inventors: Philip Wahl, Frederiksberg (DK); Henrik Vissing, Virum (DK); Christian Grondahl, Vaerlose (DK)

Correspondence Address: Novo Nordisk Pharmaceuticals, Inc.;Attention: Patent Department, 100 College Road West, Princeton, NJ, 08540, US

(21) Appl. No.: 09/934,948

(22) Filed: Aug. 22, 2001

Related U.S. Application Data
(60) Provisional application No. 60/231,670, filed on Sep. 11, 2000.

(30) Foreign Application Priority Data
Aug. 25, 2000 (DK) PA 2000 01259

(51) Int. Cl. C12P 21/06; C07H 21/04; C07K 14/72

(52) U.S. Cl. 435/69.1; 435/320.1; 435/325; 530/350; 536/23.5

(57) ABSTRACT

Proteins capable of binding meiosis activating sterols (MAS), designated SAM1a and SAM1b, and useful in methods for assaying MAS receptor ligands, agonists and antagonists, as well as DNA sequences encoding such proteins.

### FIELD OF THIS INVENTION

The present invention relates to receptors or signalling proteins of FF-MAS, polynucleotides coding for receptors or signalling proteins of FF-MAS, probes hybridising with nucleic acids encoding receptors or signalling proteins of FF-MAS, DNA constructs comprising a sequence encoding receptors or signalling proteins of FF-MAS, culture cell lines wherein the DNA sequence encodes receptors or signalling proteins of FF-MAS, antibodies specifically binding to receptors or signalling proteins of FF-MAS, hybridoma producing monoclonal antibodies specifically binding to receptors or signalling proteins of FF-MAS, and methods for detecting the presence of a compound having affinity to receptors or signalling proteins of FF-MAS.

### BACKGROUND OF THIS INVENTION

Since the first IVF pregnancy was delivered in 1978, this procedure has resulted in thousands of pregnancies and opened a vast new frontier of research and treatment for the infertile couples. Still, there is a significant need for improved infertility treatment modalities today. It is presumed that about one out of seven couples experience problems with sub fertility or infertility.

IVF of human oocytes has become commonly used for the treatment of female and male sub fertility. The standard IVF treatment includes a long phase of hormone stimulation of the female patient. The aspirated oocyte is subsequently fertilised in vitro and cultured. Continuous efforts have been made to optimise and simplify this procedure. Nevertheless, the overall pregnancy rate cannot be increased significantly over about 20% with the current treatment modalities. In a large European survey of IVF patients, it was found that 7.2 oocytes out of 11.5 aspirated oocytes per patient had undergone resumption of meiosis immediately before fertilisation, only 4.3 oocytes were fertilised and only 2.2 oocytes reached the 8-cell embryo stage after fertilisation and in vitro culture (ESHRE, Edinburgh, 1997).

Due to the very unpredictable quality of the state of the art embryos today, more than one embryo has to be transferred just to give a reasonable chance of success. Therefore, it is common to transfer 2-3 embryos (up to 5 embryos in some countries), which carries the very large side effect of multiple pregnancies with great discomfort and risk to both patient and children. Moreover, it has been estimated that the increased health care expenses due to multiple birth (twins, triplets etc.) is exceeding the entire IVF expenses. Hence, there are several disadvantages with the current treatment. Further, weight gain, bloating, nausea, vomiting, labile mood and other patient discomforts together with patient reluctance to inject themselves are reported as disadvantages.

It is known from WO 96/00235 that certain sterol derivatives can be used for regulating meiosis. An example of such a sterol is 4,4-dimethyl-5α-cholesta-8,14,24-triene-3β-ol (hereinafter designated FF-MAS).

Hence, at present, in vitro maturation in humans has proven highly unsuccessful despite substantial interest and clinical efforts.

One way of trying to find compounds which effectively regulate meiosis is the use of pertinent receptors.

Receptors are defined as proteinaceous macromolecules that perform a signal transducing function upon ligand binding. Many receptors are located on the outer cell membrane, others are located intracellularly. The substance which is bound by the receptor is called a ligand, a term which is definitionally meaningful only in terms of its counterpart receptor. The term “ligand” does not imply any particular molecular size or other structural or compositional feature other than that the substance in question is capable of binding, cleaving or otherwise interacting with the receptor in such a way that the receptor conveys information about the presence of the ligand to a target molecule. Stated alternatively, not all substances capable of binding a receptor are ligands, but all ligands are capable of binding a receptor. Receptors do not include such substances as immunoglobulins.

Receptors are believed to function by a process variously termed activation or signal transduction. A ligand binds to the ligand binding domain in such a way that the conformation of the receptor molecule changes. This conformational change, called activation, modifies the effect of the receptor on cytoplasmic components. Among changes brought about by receptor activation are changes in or development of receptor enzymatic activity.

Signalling proteins such as cAMP, IP3, kinases, and phosphatases are proteins ubiquitously found in all tissues. These proteins cascade by various pathways, the stimulus from ligand/receptor interaction down stream to cellular events, typically changing the enzymatic activity or functional state of effector molecules.

The pharmaceutical industry in recent years has oriented its research to focus on the role of receptors in disease or injury and to design drugs, generally low molecular weight substances, that are capable of binding to the receptors. Drugs identified in this initial screen are then tested for the activity in vivo or in tissue explants. As a result, conventional techniques do not lend themselves to large-scale screening. Tissue samples or isolated cells containing the target receptors, for example ovarian tissue, are costly to obtain, present in limited quantity, and difficult to maintain in a functionally viable state. Additionally, it is often difficult to reliably and reproducibly administer the candidate drug to tissue samples. Screening assays using primary explants in tissue culture are undertaken in larger scale than is possible with tissue samples. However, it is more difficult to assay physiological effect and the assays are subject to interference from many sources, for example culture media or cultivation conditions. Finally, assays using receptors isolated from natural materials have the disadvantage that the receptor is subject to natural variability and suitable natural sources may not always be available. It is an object herein to provide readily reproducible, simple assay systems that can be practiced on a large scale for determining not only ligand binding but also the character of the binding as agonistic or antagonistic.

Similarly, meaningful clinical diagnosis often depends upon the assay of biologically active ligand without interference from inactive forms of the ligand, for example, ligands that have been subject to enzymatic or other processes in the test subject that change or even eliminate the activity of the ligand. Immunoassay methods are widely used in determining ligands in test samples. However, it is often quite difficult to identify antibodies that are able to discriminate between the active and inactive forms of a ligand. Receptors have frequently been used in place of antibodies as analyte binding reagents. However, not all substances that bind to receptors are necessarily capable of inducing receptor activity, i.e., active biologically. It is an object herein to provide a method that will identify ligands in clinical test samples which are active in inducing or inhibiting signal transduction by their receptors.

Cytoplasmic proteins can act as receptors or signalling molecules in cascading the stimulus from the ligand to cellular events. Various receptors or signalling protein types make use of different path ways (for example small G proteins, calcium fluxes, phospatases, and lipases), all of them resulting in changes of enzymatic activity or gene transcription.

Meiotic activating sterols (hereinafter designated MAS) constitute active signalling molecules first identified in follicular fluid and in bull testicular tissue. The sterols are described by Byskov 1995 and Grøndahl et al. (Biol. Reprod. 58 (1998), 1297 et seq.) and in WO 96/00235, 96/27658, 97/00884, 98/28323, 98/54965 and 98/55498, more specifically in claim 1 thereof, as being potent activators of the meiotic process. No receptors or signalling proteins have been described to directly or indirectly signalling the meiotic effect of MAS sterols. Before this invention, the presence of the nature of a putative MAS receptor protein or a signalling protein has not previously been identified, although its presence has been suggested, for example, by Grøndahl et al. (Biol. Reprod. 62 (2000), 775 et seq.).

On 29 Sep. 2000, the nucleotide and amino acid sequence of clone NT2RM2001632 was released with accession number AK022554 and on 10 May 2001, the nucleotide and amino acid sequence of clone NT2RP2000448 was submitted with accession number AK027535. No utility or action was mentioned for these clones.

There remains considerable need for an isolated and purified MAS receptor or a MAS signalling protein, as well as systems capable of expressing a MAS receptor or a MAS signalling protein separate from other receptors. Further, it would be desirable to specifically identify the presence of a MAS receptor or a MAS signalling protein in cells and tissues, thereby avoiding the time-consuming, complex and non-specific functional pharmacological assays. It would also be desirable to screen and develop new agonists and/or antagonists specific for a MAS receptor or a MAS signalling protein for the use of antiinfertility or contraception drugs, but to date this has not been possible. Quite surprisingly, the present invention fulfils these and other related needs.

### SUMMARY OF THE INVENTION

The present invention provides the nucleotide sequence of the receptors or signalling proteins of meiotic acting sterols (MAS). The present invention provides isolated and substantially pure MAS receptors or MAS signalling proteins and fragments thereof. These receptors or signalling proteins have been shown to be involved in the gamete maturation process induced by 3β-hydroxy-4,4-dimethylcholest-8,14,24-triene (hereinafter designated FF-MAS), specifically inducing, upon ligand activation, germinal vesicle breakdown (hereinafter designated GVB) in mouse oocyte cultured in vitro. Hence, a MAS receptor or a MAS signalling protein is defined as a proteinaceous macromolecule that perform a signal transducing function upon binding to FF-MAS. Briefly, a MAS receptor or a MAS signalling protein binds to FF-MAS. Alternatively, a MAS signalling protein can be designated a MAS binding protein.

A MAS receptor is any protein related to the sterol activated molecule (SAM) protein SAM1a or SAM1b that possess the same functional characteristic regarding the interaction with FF-MAS or other endogenous meiosis activating sterols, for example, 3β-hydroxycholest-8,14-diene; 3β-hydroxy-4,4-dimethylcholest-8,24-diene; and 3β-hydroxycholest-8,24-diene, or their metabolites (as ligand). Functional characteristics include binding, receptor activation, and subsequent germinal vesicles breakdown (GVB) in oocytes. The amino acid sequence of SAM1a and SAM1b is stated in SEQ ID NO: 2 and SEQ ID NO: 4, below.

The MAS receptor can be used to discover profertility and antifertility compounds which can be used to men and women.

Having provided such receptors or signalling proteins in isolated or purified form, the invention also provides antibodies to the MAS receptor or signalling protein, in the form of antisera and/or monoclonal antibodies.

In another aspect, the invention provides the ability to produce the MAS receptor or MAS signalling protein and polypeptides or fragments thereof by recombinant means. The expressed MAS receptor or signalling protein or fragments may or may not have the biological activity of native receptor or signalling protein. Accordingly, isolated and purified polynucleotides are described which code for the receptor or signalling protein and fragment thereof, where the polynucleotides may be in the form of DNA, such as cDNA, or RNA. Based on these sequences, probes may be used to hybridise and identify these and related genes which encode MAS receptors or MAS signalling proteins. The probes may be full length cDNA or as small as form 14 to 25 nucleotide, more often though from about 40 to about 50 or more nucleotides.

In related embodiments, the invention concerns DNA constructs which comprise a transcriptional promoter, a DNA sequence which encodes the receptor or signalling protein or fragment, and a transcriptional terminator, each operably linked for expression of the receptor or signalling protein. For expression, the construct may also contain at least one signal sequence. Further, for large-scale production, the expressed receptor or signalling protein may also be isolated from the cells by, for example, immunoaffinity purification.

Cells or bacteria which express the MAS receptor or MAS signalling proteins may also be used to identify compounds which can alter the receptor or signalling protein-mediated metabolism of a cell. Compounds may be screened for binding to the receptor or signalling protein, and/or for effecting a change in receptor or signalling protein-mediated metabolism in the host cell. Agonists and/or antagonists of the MAS receptor or MAS signalling proteins may also be screened in cell-free systems using purified receptor or signalling proteins or binding fragments thereof for the effect on ligand/receptor interaction or ligand/signalling protein interaction, or using reconstituted systems such as micelles which also provide the ability to assess metabolic changes.

In yet other embodiments, the invention relates to methods for diagnosis, where the presence of a mammalian MAS receptor or MAS signalling protein in a biological sample may be determined. For example, a monospecific antibody which specifically binds the receptor or signalling protein is incubated with the sample under conditions conducive to immune complex formation, which complexes are then detected, typically by means of a label such as an enzyme, fluorophore, radionuclide, chemiluminiscer, particle, or a second labelled antibody. Thus, means are provided for immunohistochemical staining of tissues, including ovarian or testicular tissues, for the subject receptor or signalling proteins.

Based upon the similarity in sequence and the shared presence of a sterol binding domain at the protein level, the receptor proteins or signalling proteins of this invention can be said to belong to a novel super family of oxysterol binding proteins (hereinafter designated OSPB) recently published in J.Lipid.Res. 40 (1999), 2204. No function whatsoever in gamete maturation of either gender or regulation of any meiotic processes has been assigned to this OSPB family.

### BRIEF DESCRIPTION OF THE FIGURES

FIG. 1 shows the displacement of 3H-labelled FF-MAS from SAM1b as described below (Example 2).

### DESCRIPTION OF THE PREFERRED EMBODIMENTS

The present invention presents the means to identify agonists, and antagonists of the MAS receptor/ligand interaction or MAS signalling protein/ligand interaction by providing isolated MAS receptor or MAS signalling protein. The term “MAS receptor” refers to any proteins either derived from a naturally occurring MAS receptor, or which shares significant structural and functional characteristics peculiar to a naturally occurring MAS receptor. Such a receptor may result when regions of a naturally occurring receptor are deleted or replaced in such a manner as to yield a protein having a similar function. Homologous sequences, allelic variations, and natural mutants; induced point, deletion, and insertion mutants; alternatively expressed variants; proteins encoded by DNA which hybridise under high or low stringency conditions to nucleic acids which encode naturally occurring MAS receptor; proteins retrieved from naturally occurring materials; and closely related proteins retrieved by antisera directed against MAS receptor proteins are also included. Similarly, this applies to MAS sig-signalling proteins.

By MAS receptor “ligand” is meant a molecule capable of being bound by the ligand-binding domain of MAS receptor, a MAS receptor analogue, or chimeric MAS receptor as generally described in U.S. Pat. No. 4,859,609, incorporated by reference herein. The molecule may be chemically synthesised or may occur in nature. Ligands may be grouped into agonists and antagonists. Agonists are those molecules whose binding to a receptor induces the response pathway within a cell. Antagonists are those molecules whose binding to a receptor blocks the response pathway within a cell.

By “isolated” MAS receptor or MAS signalling protein is meant to refer to MAS receptor or MAS signalling protein which is in other than its native environment such as a mammalian oocyte, including, for example, substantially pure MAS receptor as defined herein below. More generally, isolated is meant to include MAS receptor or MAS signalling protein as a heterologous component of a cell or other system. For example, MAS receptor or MAS signalling protein may be expressed by a cell transfected with a DNA construct which encodes MAS receptor or MAS signalling protein, separated from the cell and added to micelles which contain other selected receptor or signalling proteins. In some instances, the term MAS receptor covers both a MAS receptor and a MAS signalling protein.

By purified MAS receptor or MAS signalling protein is meant MAS receptor or MAS signalling protein having a purity of at least 50%, preferably at least 80%, more preferred at least 90% (w/w).

Human sterol activated molecule 1a (SAM1a) and SAM1 b are the clones NT2RP2000448 and NT2RM2001632, respectively.

A similar way of defining the MAS receptors or MAS signalling proteins of the present invention is the similarity between the amino acid sequence of the various MAS receptors or MAS signalling proteins is at least about 90%, preferably about 95%, when compared with the amino acid sequence in SEQ ID NO: 2. Another expression of amino acid sequence similarity is homology. At the nucleotide level, the similarity between the nucleotides of the various MAS receptors or MAS signalling proteins is at least about 80%, preferably about 90%, when compared with the nucleotides in SEQ ID NO: 1.

One way of determining the affinity constant of a MAS receptor or a MAS signalling protein is by the test described in example 3, below. One way of determining whether FF-MAS binds to a specific MAS receptor is by the test described in example 4, below. The test described in example 3 below can be used to determine whether a specific protein is a MAS receptor or a MAS signalling protein or, in other words, whether a specific protein is an analogue of SAM1a as specified in the claims below. In the present context, the term “analogue” is intended to indicate a naturally occurring variant (including one expressed in other animal species, for example, human, monkey, mouse or rat) of the MAS receptor or a “derivative”, i.e., a polypeptide which is derived from the native MAS receptor by suitably modifying the DNA sequence coding for the variant, resulting in the addition of one or more amino acids at either or both the C- and N-terminal ends of the native amino acid sequence, substitution of one or more amino acids at one or more sites in the native amino acid sequence, deletion of one or more amino acids at either or both ends of the native sequence or at one or more sites within the native sequence, or insertion of one or more amino acids in the native sequence.

In another aspect, the invention provides means for regulating the MAS receptor/ligand interaction or MAS signalling protein/ligand interaction, and thus treating, therapeutically and/or prophylactically, a disorder which can be linked directly or indirectly to MAS receptor or MAS signalling protein or to its ligands, such as FF-MAS. By virtue of having the receptor or signalling protein of the invention, agonists or antagonists may be identified which stimulate or inhibit, respectively, the interaction of ligand with MAS receptor or with MAS signalling protein. With either agonists or antagonists, the metabolism and reactivity of cells which express the receptor or signalling protein are controlled, thereby providing a means to control meiosis in order to treat infertility or to achieve a novel principle of contraception.

Thus, the invention provides screening procedures for identifying agonists or antagonists of events mediated by the ligand/MAS receptor or ligand/MAS signalling protein interaction. Such screening assays may employ a wide variety of formats, depending to some extent on which aspect of the ligand, receptor or signalling protein interaction is targeted. For example, such assays may be designed to identify compounds which bind to the receptor or signalling protein and thereby block or inhibit interaction of the receptor or signalling protein and thereby block or inhibit interaction of the receptor or signalling protein with the ligand. Other assays can be designed to identify compounds which can substitute for ligand and therefore stimulate MAS receptor-mediated or MAS signalling protein-mediated intracellular pathways. Yet other assays can be used to identify compounds which inhibit or facilitate the association of MAS receptor or MAS signalling protein to FF-MAS and thereby mediate the cellular response to MAS receptor or MAS signalling protein ligand.

In one functional screening assay, the initiation of fertilisation activation events are monitored in eggs which have been injected with, for example, mRNA which codes for MAS receptor or MAS signalling protein and subsequently exposed to selected compounds which are being screened, in conjunction with or apart form an appropriate ligand. See generally, Kline et al., Science 241 (1988), 464-467, incorporated herein by reference.

The screening procedure can be used to identify reagents such as antibodies which specifically bind to the receptor or signalling protein and substantially affect its interaction with ligand, for example. The antibodies may be monoclonal or polyclonal, in the form of antiserum or monospecific antibodies, such as purified antiserum or monoclonal antibodies or mixtures thereof. For administration to humans, for example, as a component of a composition for in vivo diagnosis or imaging, the antibodies are preferably substantially human to minimise immunogenicity and are in substantially pure form. By substantially human is meant generally containing at least about 70% human antibody sequence, preferably at least about 80% human, and most preferably at least about 90-95% or more of a human antibody sequence to minimise immunogenicity in humans.

Antibodies which bind to a MAS receptor or a MAS signalling protein may be produced by a variety of means. The production of non-human antisera or monoclonal antibodies, for example, murine, lagomorpha equine, etc. is well known and may be accomplished by, for example, immunising the animal with the receptor or signalling protein molecule or a preparation containing a desired portion of the receptor or signalling protein molecule, such as that domain or domains which contributes to ligand binding. For the production of monoclonal antibodies, antibody-producing cells obtained from immunised animals are immortalised and screened, or screened first for the production of antibody which binds to the receptor or signalling protein and then immortalised. As the generation of human monoclonal antibodies to human MAS receptor or MAS signalling protein antigen may be difficult with conventional techniques, it may be desirable to transfer antigen binding regions of the non-human antibodies, for example, the F(ab′)₂ or hypervariable regions, to human constant regions (Fc) or framework regions by recombinant DNA techniques to produce substantially human molecules. Such methods are generally known in the art and are described in, for example, U.S. Pat. No. 4,816,397, and EP publications 173,494 and 239,400, which are incorporated herein by reference. Alternatively, one may isolate DNA sequences which code for a human monoclonal antibody or portions thereof that specifically bind to the human receptor or signalling protein by screening a DNA library from human B cells according to the general protocol outlined by Huse et al., Science 246 (1989), 1275-1281, incorporated herein by reference, and then cloning and amplifying the sequences which encode the antibody (or binding fragment) of the desired specificity.

In other embodiments, the invention provides screening assays conducted in vitro with cells which express the receptor or signalling protein. For example, the DNA which encodes the receptor or signalling protein or selected portions thereof may be transfected into an established cell line, for example, a mammalian cell line such as BHK and CHO, using procedures known in the art (see, for example, Sambrook et al., Molecular Cloning, A Laboratory Manual, 2^{nd} edition, Cold Spring Harbor Laboratory Press, Cold Spring Harbor, N.Y., 1989, which is incorporated herein by reference). The receptor or signalling protein is then expressed by the cultured cells, and selected agents are screened for the desired effect on the cell, separately or in conjunction with an appropriate ligand such as FF-MAS or other MAS compounds. Means for amplifying nucleic acid sequences which may be employed to amplify sequences encoding the receptor or signalling protein or portions thereof are described in U.S. Pat. Nos. 4,683,195 and 4,683,202, incorporated herein by reference.

In yet another aspect, the screening assays provided by the invention relate to transgenic mammals whose germ cells and somatic cells contain a nucleotide sequence encoding MAS receptor protein or signalling protein or a selected portion of the receptor or signalling protein which, for example, binds ligand. In yet a further aspect, the screening assays provided by the invention relate to transgenic mammals where the nucleotide sequence encoding a MAS receptor or a MAS signalling protein is molecularly targeted to produce knock out animals with the phenotypical loss of the specific MAS signalling function. Preferentially, the molecular knock out is tissue specific to gonadal tissue (ovary or testes) and is timely controlled in the development, thus inducible. There are several means by which a sequence encoding, for example, the human MAS receptor may be introduced into a non-human mammalian embryo or, alternatively, knocked out, some of which are described in, for example, U.S. Pat. No. 4,736,866, Jaenisch, Science 240: 1468-1474 (1988) and Westphal et al., Annu. Rev. Cell Biol. 5: 181-196 (1989), which are incorporated herein by reference. The animal's cells then express the receptor or signalling protein and thus may be used as a convenient model for testing or screening selected agonists or antagonists.

In another aspect the invention concerns diagnostic methods and compositions. By means of having the MAS receptor or MAS signalling protein molecule and antibodies thereto, a variety of diagnostic assays are provided. For example, with antibodies, including monoclonal antibodies, to MAS receptor or MAS signalling protein, the presence and/or concentration of receptor or signalling protein in selected cells or tissues in an individual or culture of interest may be determined. These assays can be used in the diagnosis and/or treatment of diseases such as, for example, male infertility, female infertility, or by means of contraception in both gender.

Numerous types of immunoassays are available and are known to those skilled in the art, for example, competitive assays, sandwich assays, and the like, as generally described in, for example U.S. Pat. Nos. 4,642,285; 4,376,110; 4,016,043; 3,879,262; 3,852,157; 3,850,752; 3,839,153; 3,791,932; and Harlow and Lane, Antibodies, A Laboratory Manual, Cold Spring Harbor Publications, N.Y. (1988), each incorporated by reference herein. In one assay format, MAS receptor or MAS signalling protein is identified and/or quantified by using labelled antibodies, preferably monoclonal antibodies which are reacted with brain tissues, for example, ovarian or testicular tissue, oocyte preparations, or semen samples, and determining the specific binding thereto, the assay typically being performed under conditions conducive to immune complex formation. Unlabeled primary antibody can be used in combination with labels that are reactive with primary antibody to detect the receptor or signalling protein. For example, the primary antibody may be detected indirectly by a labelled secondary antibody made to specifically detect the primary antibody. Alternatively, the anti-MAS receptor-antibody or MAS signalling protein-antibody can be directly labelled. A wide variety of labels may be employed, such as radionuclides, particles (for example, gold, ferritin, magnetic particles, red blood cells), flourophores, chemiluminescers, enzymes, enzyme substrates, enzyme cofactors, enzyme inhibitors, and ligands (particularly haptens).

The RNA encoding the MAS receptor MAS signalling protein may be directly detected in cells with a labelled synthetic oligonucleotide probe targeting the MAS receptor RNA or MAS signalling protein RNA in a hybridisation procedure. Also, the polymerase chain reaction (Saiki et al., Science 239 (1988), 487, and U.S. Pat. No. 4,683,195, each reference is hereby incorporated by reference) may be used to amplify DNA sequences, which are subsequently detected by their characteristic size on agarose gels, Southern blot of these gels using MAS receptor DNA or MAS signalling protein DNA or a oligonucleotide probe, or a dot blot using similar probes. The probes may comprise from about 14 nucleotides to about 25 or more nucleotides, preferably, 40 to 60 nucleotides, and in some instances a substantial portion or even the entire cDNA of MAS receptor or MAS signalling protein may be used. The probes are labelled with detectable signal, such as an enzyme, biotin, a radionuclide, fluorophore, chemiluminescer, and paramagnetic particle. High stringency in connection with hybridisation is obtained using the proper temperature and salt concentration.

By the term “high stringency” conditions is meant conditions under which the labelled probe encoding part or all of a MAS receptor will hybridize with high specificity to polynucleotide sequences containing few, if any, mismatched bases. High-stringency hybridization conditions are described in, for example, Sambrook et al. 1989, “Molecular Cloning”, Cold Spring Harbor Laboratory Press.

In the case of Sam1a or Sam1b probes in the form of polynucleotides of 200 bases or more, high stringency hybridization is achieved by incubating the probe and the membrane containing target DNA or mRNA in a buffer containing 6×SSC, 10% Dextran sulphate, 1% SDS, 5× Denhardts, 50 ug/ml salmon DNA (Stratagene), and 2×10E6 cpm/ml of the radiolabelled probe. The incubation is at 68° C. with shaking or rotation for at least 2 hours, typically overnight. The membrane is then washed in 2×SSC, 0,1% SDS at 42° C. for 30 min, followed by a wash in 2×SSC, 0,1% SDS at 68° C. for 30 min, then a wash in 0,2×SSC, 0,1% SDS at 68° C., and finally a wash in 0,1×SSC, 0,1% SDS at 68° C. for 30 min. The membrane is exposed to x-ray film.

In case of oligonucleotide probes 25-200 bases in length, high stringency hybridization is carried out in a solution containing 6×SSC, 0,05 M sodium phosphate (pH 6,8), 1 mM EDTA (pH 8,0), 5× Denhardts solution, 100 ug/ml salmon sperm DNA, 100 mg/ml dextran sulfate, and 180 pM of radiolabelled oligonucleotide (5×10E5 to 1,5E6 cpm/pmole). The hybridization temperature vary depending on the length of the probe. Sambrook et al. (supra, chapter 11) have described how to calculate, and experimentally verify, the conditions that will result in high-stringency hybridization. Hybridization is performed at 5-10 degrees less than the Tm, and post-hybridization washes at 5 degrees below the Tm, with the Tm calculated as
Tm=81,5−16,6(Log10[Na+])+0,41(% G+C)−(600/N), where N=chain length.

Hybridization is done overnight with shaking or rotation. The membrane is then washed twice with 2×SSPE, 0,1% SDS at room temperature for 15 minutes, then with 0,2×SSPE, 0,1% SDS 5 degrees below the Tm of the probe, for 60 minutes. The membrane is exposed to x-ray film.

Kits can also be supplied for use with the receptor or signalling protein of the subject invention in the detection of the presence of the receptor or signalling protein or antibodies thereto, as might be desired in the case of autoimmune disease. Thus, antibodies to MAS receptor or MAS signalling protein, preferably monospecific antibodies such as monoclonal antibodies, or compositions of the receptor or signalling protein may be provided, usually in lyophilised form in a container, either segregated or in conjunction with additional reagents, such as anti-antibodies, labels, gene probes, polymerase chain reaction primers and polymerase, and the like.

Even more specifically, the present invention relates to an isolated and/or purified polynucleotide molecule which hybridises at high stringency to an oligonucleotide of 25 or more contiguous nucleotides of SEQ ID NO: 1 or SEQ ID NO: 3 and which polynucleotide codes for a) a MAS receptor or for a MAS signalling protein; or b) a ligand binding domain of a MAS receptor or MAS signalling protein. This polynucleotide may be a RNA antisense sequence or a cDNA sequence. This polynucleotide may encode a polypeptide displaying MAS receptor or MAS signalling protein activity. This polynucleotide may encodes a MAS receptor or MAS signalling protein (being able to bind to FF-MAS) having the amino acid sequence of SEQ ID NO: 2 or SEQ ID NO: 4. The polynucleotide may have the nucleotides of SEQ ID NO: 1 or SEQ ID NO: 3.

Furthermore, this invention relates to a probe of at least 12 nucleotides, said probe being capable of hybridising with nucleic acids which encode a MAS receptor or MAS signalling protein. This probe may comprise an oligonucleotide or polynucleotide of 25 or more contiguous nucleotides of SEQ ID NO: 1 or SEQ ID NO: 3 capable of specifically hybridising with a gene which encodes a MAS receptor or MAS signalling protein, or allelic and species variants thereof. This probe may comprise from about 40 to about 60 nucleotides in length. This probe may be labelled to provide a detectable signal. This probe may comprise the nucleotides of SEQ ID NO: 1 or SEQ ID NO: 3.

Furthermore, the present invention relates to a DNA construct comprising a DNA sequence which hybridises at high stringency to an oligonucleotide or polynucleotide of 25 or more contiguous nucleotides of SEQ ID NO: 1 or SEQ ID NO: 3 and which encodes a) a MAS receptor or MAS signalling protein; or b) a ligand binding domain of a MAS receptor or MAS signalling protein. This DNA construct may have a DNA sequence encoding a MAS receptor or MAS signalling protein having the amino acid sequence of SEQ ID NO: 2 or SEQ ID NO: 4.

Furthermore, the present invention relates to a cultured cell line, yeast or bacteria transformed or transfected with a DNA construct which comprises a DNA sequence which hybridises at high stringency to an oligonucleotide or polynucleotide of 25 or more contiguous nucleotides of SEQ ID NO: 1 or SEQ ID NO: 3 and which encodes a) a MAS receptor or MAS signalling protein; or b) a ligand binding domain or a transmembrane domain of a MAS receptor or MAS signalling protein. This cell line, yeast or bacteria may not express endogenous MAS receptor or MAS signalling proteins.

The MAS receptor or MAS signalling protein, a peptide fragment thereof or a salt thereof according to the present invention may be isolated and/or purified. The isolated and/or purified protein (MAS receptor or MAS signalling protein) may comprise the amino acid sequence of SEQ ID NO: 2 or SEQ ID NO: 4.

Furthermore, the present invention relates to an isolated antibody which specifically binds to a MAS receptor or MAS signalling protein. In this isolated antibody said antibody may be a monoclonal antibody. This isolated antibody may block the binding of MAS to a MAS receptor or MAS signalling protein.

Furthermore, the present invention relates to a hybridoma which produces a monoclonal antibody as mentioned herein.

Furthermore, the present invention relates to a method for detecting the presence of a compound or a salt thereof which has affinity for a MAS receptor or MAS signalling protein, comprising the steps of a) contacting the compound with the MAS receptor or MAS signalling protein, a peptide fragment thereof or a salt thereof; and b) measuring the affinity of said compound for the MAS receptor or MAS signalling protein. This method for detecting the presence of MAS antagonists, may comprise the steps of a) exposing a compound in the presence of a MAS agonist including MAS (FF-MAS) to a MAS receptor or MAS signalling protein coupled to a response pathway under conditions and for a time sufficient to allow binding of the compound to the MAS receptor or MAS signalling protein and an associated response through the pathway; and b) detecting a reduction in the stimulation of the response pathway resulting from the binding of the compound to the MAS receptor or MAS signalling protein, relative to the stimulation of the response pathway by the MAS agonist alone and there from determining the presence of a MAS antagonist. Furthermore, a method for detecting the presence of MAS agonists, may comprise the steps of a) exposing a compound in the presence of a MAS antagonist to a MAS receptor or MAS signalling protein coupled to a response pathway under conditions and for a time sufficient to allow binding of the compound to the MAS receptor or MAS signalling protein and an associated response through the pathway; and b) detecting an increase of the stimulation of the response pathway resulting from the binding of the compound to the MAS receptor or MAS signalling protein, relative to the stimulation of the response pathway by the MAS antagonist alone and there from determining the presence of a MAS agonist.

Furthermore, the present invention relates to a compound or a salt thereof which has affinity for the MAS receptor or a MAS signalling peptide and which compound or salt is detected by a method described herein.

Furthermore, the present invention relates to a method for producing a MAS receptor or MAS signalling protein having the amino acid sequence of SEQ ID NO: 2 or SEQ ID NO: 4, which may comprise a) growing cells, yeast or bacteria transformed or transfected with a DNA construct which comprises a DNA sequence of SEQ ID NO: 1 or SEQ ID NO: 3 coding for the expression of the MAS receptor or MAS signalling protein, and b) isolating the MAS receptor or MAS signalling protein from the cells. In this method, the MAS receptor or MAS signalling protein may be isolated by immunoaffinity purification.

Furthermore, the present invention relates to a kit for screening a compound or a salt thereof which has affinity for a MAS receptor or MAS signalling protein, which contains the MAS receptor or MAS signalling protein, the peptide fragment thereof or a salt thereof.

Alternatively, the MAS receptor or MAS signalling protein may be defined as one which comprises the amino acid sequence shown in SEQ ID NO: 2, or an analogue thereof binding FF-MAS, with an affinity constant below 100 μM, preferably below 10 μM. The MAS receptor or MAS signalling protein comprising the amino acid sequence shown in SEQ ID NO: 2, or an analogue thereof binding FF-MAS, with an affinity constant below 100 μM, preferably below 10 μM, may be different from the amino acid sequence in SEQ ID NO: 6 and 8. Alternatively, the MAS receptor or MAS signalling protein may comprise the amino acid sequence shown in SEQ ID NO: 4, or an analogue thereof binding FF-MAS, with an affinity constant below 100 μM, preferably below 10 μM. The MAS receptor or MAS signalling protein comprising the amino acid sequence shown in SEQ ID NO: 4, or an analogue thereof binding FF-MAS, with an affinity constant below 100 μM, preferably below 10 μM, may be different from the amino acid sequence in SEQ ID NO: 6 and 8. Alternatively, the MAS receptor or MAS signalling protein may comprise the partial amino acid sequence shown in SEQ ID NO: 6, or an analogue thereof binding FF-MAS, with an affinity constant below 100 μM, preferably below 10 μM. The MAS receptor or MAS signalling protein comprising the partial amino acid sequence shown in SEQ ID NO: 6, or an analogue thereof binding FF-MAS, with an affinity constant below 100 μM, preferably below 10 μM, may be different from the amino acid sequence in SEQ ID NO: 6 and 8. Alternatively, the MAS receptor or MAS signalling protein may comprise the partial amino acid sequence shown in SEQ ID NO: 8, or an analogue thereof binding FF-MAS, with an affinity constant below 100 μM, preferably below 10 μM. The MAS receptor or MAS signalling protein comprising the partial amino acid sequence shown in SEQ ID NO: 8, or an analogue thereof binding FF-MAS, with an affinity constant below 100 μM, preferably below 10 μM, may be different from the amino acid sequence in SEQ ID NO: 6 and 8. The MAS receptor or MAS signalling protein according to the present invention may be a soluble and purified protein which is present in a buffer suitable for detecting ligands, for example by a binding assay. The MAS receptor or MAS signalling protein being a soluble and purified protein which is present in a buffer suitable for detecting ligands, for example by a binding assay, may be different from the amino acid sequence in SEQ ID NO: 6 and 8.

Furthermore, the present invention relates to a DNA construct which comprises a DNA sequence encoding a MAS receptor or MAS signalling protein as described herein or a DNA sequence coding for a functional analog thereof binding to FF-MAS. The DNA construct comprises a DNA sequence encoding a MAS receptor or MAS signalling protein as defined herein or a DNA sequence coding for a functional analog thereof binding to FF-MAS may be different from the nucleotides of SEQ ID NO: 5 and 7. The DNA construct of the present invention may comprise the DNA sequence shown in SEQ ID NO: 1 or a fragment thereof, or a DNA sequence coding for a functional analogue thereof binding FF-MAS, with an affinity constant below 100 μM, preferably below 10 μM. This DNA construct comprising the DNA sequence shown in SEQ ID NO: 1 or a fragment thereof, or a DNA sequence coding for a functional analogue thereof binding FF-MAS, with an affinity constant below 100 μM, preferably below 10 μM, may be different from the nucleotides of SEQ ID NO: 5 and 7. The DNA construct according to the present invention may comprise the partial DNA sequence shown in SEQ ID NO: 5, or a DNA sequence coding for a functional analogue thereof binding FF-MAS, with an affinity constant below 100 μM, preferably below 10 μM. This DNA construct comprising the partial DNA sequence shown in SEQ ID NO: 5, or a DNA sequence coding for a functional analogue thereof binding FF-MAS, with an affinity constant below 100 μM, preferably below 10 μM, may be different from the nucleotides of SEQ ID NO: 5 and 7.

Furthermore, the present invention relates to a recombinant expression vector which carries an inserted DNA construct according to any one of the preceding claims to a DNA construct.

Furthermore, the present invention relates to a cell containing a recombinant expression vector as defined herein. This cell may contain a DNA construct as defined herein integrated in its genome. This cell may be a eukaryotic cell, in particular an insect or a mammalian cell.

Furthermore, the present invention relates to a method of screening for ligands to the MAS receptor, i.e., agonists or antagonists of FF-MAS activity, the method comprising incubating a MAS receptor or MAS signalling protein as defined herein with a substance suspected to be an agonist or antagonist of FF-MAS, and subsequently with FF-MAS, or an analogue thereof, and detecting any effect of binding of FF-MAS, or the analogue to the MAS receptor or MAS signalling protein. Alternatively, the method of screening for ligands to the MAS receptor, i.e., agonists or antagonists of FF-MAS activity, may comprise incubating FF-MAS, or an analogue thereof with a substance suspected to be an agonist or antagonist of activity of FF-MAS, and subsequently with a MAS receptor or MAS signalling protein as described herein, and detecting any effect of binding of FF-MAS, or the analogue to the receptor.

Furthermore, the present invention relates to the use of a MAS receptor or MAS signalling protein as defined herein for screening for agonists or antagonists of activity of FF-MAS.

Furthermore, the present invention relates to the use of DNA constructs as defined herein for isolation of tissue and/or organ specific variants of the MAS receptor or MAS signalling protein.

Furthermore, the present invention relates to the use of a MAS receptor or MAS signalling protein isolated as described herein.

### EXAMPLES

The following examples are offered by way of illustration, not by limitation.

### EXAMPLE 1

### Microinjection of Phosphorothionate Oligonucleotides into Mouse Oocytes

Two antisense oligonucleotides (20 nucleotides) were utilized for microinjection: 5′-TCCACGATGGACGCCATCTT-3′ and 5′-GCCAGCAGGAGAGCCATTCG-3′, complementary to the kozak sequence of the mRNA encoded by the cDNA sequence herein designated SAM1a and SAM1b, respectively, both of which are defined in SEQ ID NO: 1 and SEQ ID NO: 3, respectively, shown below. In control experiments, the corresponding sense oligonucleotides were microinjected: 5′-AAGATGGCGTCCATCGTGGA-3′ and 5′-CGAATGGCTCTCCTGCTGGC-3′ for mRNA SAM1a and SAM1b, respectively. SAM1a antisense was co-injected with SAM1b antisense from a stock solution containing 1.25 μg/μl of each nucleotide in 10% human serum albumin (hereinafter designated HSA) plus 5 mM Tris (pH value: 7.5). SAM1a sense was co-injected with SAM1b sense from a stock solution containing 1.25 μg/μl of each nucleotide in 10% HSA plus 5 mM Tris (pH value: 7.5). Approximately 12 pg of each oligonucleotide (10 μl) were injected into the cytoplasma of individual germinal vesicle (GV)-stage oocytes loaded in a droplet of alpha-MEM supplemented with 0.8% HSA and 3 mM hypoxantine under mineral oil in a 35 mm petri dish on the stage of an inverted microscope. The oocytes were obtained from the ovaries of 21-24 days old mice following 48 hours priming with follicle stimulating hormone (hereinafter designated FSH) as described by Grøndahl et al. 1998 in Biol. Reprod. 58 (1998), 1297 et seq. Oocytes were sucked on to a holding pipette (120 μM outer diameter and 20 μm inner diameter) and an injection pipette (Eppendorph, Hamburg, Germany) was fitted to a pressure microinjector (Eppendorph, Hamburg, Germany). The pipette holder was attached to a piezoelectric positioning system (Burleigh, N.Y., USA) mounted on a motorized micromanipulator (Luigs and Neumann, Ratingen, Germany). The injection pipette was pushed against the zona pelludica, and then a piezoelectric pulse was given, moving the injection pipette 20 μm forward. During this movement the pipette penetrated the zona pelludica and oolema and then a brief pressure pulse was applied to release a volume of approximately 10 μl into the oocytes cytoplasm. Injected oocytes were placed in a CO₂ incubator at 37° C. for 20 hours before resumption of meiosis was triggered by addition of 10 μM FF-MAS to the hypoxanthine containing medium. The effect of FF-MAS was evaluated after 24 hours of further incubation as the number of oocytes in germinal vesicle breakdown (hereinafter designated GVBD). The rationale for the 20 hours cultivation period following injection of antisense oligonucleotides is to allow for degradation of mRNA coding for SAM1a and SAM1b protein. Consequently, when the level of MAS receptor protein or MAS signalling protein is reduced in the oocytes, the MAS response is blunted (from 100% to 50%, vide the table below).

TABLE 1

| | GVBD/GVBD + GV |
|---|---|
| Oligonucleotide | 10 μM FF-MAS (24 hours) |
| SAM1a + SAM1b | 13/25 |
| Antisense | |
| SAM1a + SAM1b | 10/10 |
| Sense | |
| Non-injected oocytes | 26/29 |

As shown in Table 1, GVBD was inhibited by 50% in antisense injected oocytes compared to control (i.e., sense injected and non-injected oocytes). This result indicates a selective inhibition of the mRNAs coding for SAM1a and SAM1b by the antisense probe. Furthermore, these results indicate that SAM1a and SAM1b proteins are crucial involved in the MAS signalling, since a functional knock out of de novo protein synthesis of these molecules partly disrupt the MAS signals in oocytes.

SAM1a and SAM1b are two closely related proteins originating from the same gene which possesses complementary functions regarding MAS signalling in oocytes.

### EXAMPLE 2

### FF-MAS Binding Assay

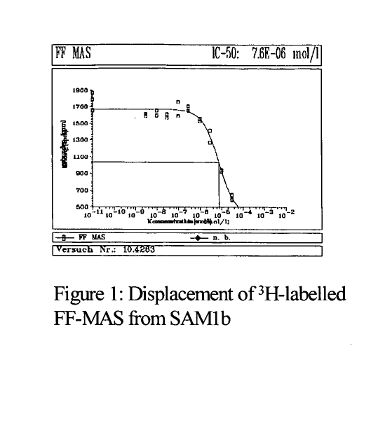

10 μl of the unlabelled FF-MAS (1, 3, 10, 30, 100, 300, 1000, 3000 nM in 6.6% ethanol (EtOH)) was mixed with 10 μl of 200 nM ³H-labelled FF-MAS (approximately 12.8 Ci/mmol) in assay buffer (10 mM Tris; 1.5 mM EDTA; 10% glycerin; 1.0 mM 3-(3-cholamidopropyl)dimethylamino-1-propanesulphonate (hereinafter designated CHAPS, Boehringer Mannheim); 1% BSA (bovine serum albumin)). 10 μg of SAM1b protein freshly diluted in assay buffer, was added to give a final volume of 40 μl. Unspecific binding was measured in the presence of 30 μM unlabelled FF-MAS, total binding was determined by adding 10 μl assay buffer containing 6.6% EtOH. Incubation was performed for 2 hours at 4° C. 250 μl of ice-cold assay buffer containing 2% Cab-osil M-5 (silica gel from Fluka) and 0.2% dextran T70 (Sigma) was added to each tube, mixed and spinned briefly. Approximately 5 minutes after adding Cab-osil M-5, the tubes were centrifuged for 5 minutes, 14000 rpm (minifuge). 200 μl of the supernatant was transferred to a microscint-tube and 3.5 ml atomlight (Packard) was added. Tubes were measured in a liquid scintillation counter. The results obtained are shown in FIG. 1. “IC-50” is the concentration of unlabelled FF-MAS which displaces 50% of the bound ³H FF-MAS.

### EXAMPLE 3

### Assay to Determine Whether a Specific Polynucleotide Encodes a Protein which is a MAS Receptor or a MAS Signalling Protein

10 μl of the unlabelled FF-MAS (1, 3, 10, 30, 100, 300, 1000, 3000 nM in 6.6% EtOH) is mixed with 10 μl of 200 nM ³H-labelled FF-MAS (approximately 12.8 Ci/mmol) in assay buffer (10 mM Tris; 1.5 mM EDTA; 10% glycerin; 1.0 mM CHAPS; 1% BSA). 10 μg of the specific protein to be tested freshly diluted in assay buffer, is added to give a final volume of 40 μl. Unspecific binding is measured in the presence of 30 μM unlabelled FF-MAS, total binding is determined by adding 10 μl assay buffer containing 6.6% EtOH. Incubation is performed for 2 hours at 4° C. 250 μl of ice-cold assay buffer containing 2% Cab-osil M-5 and 0.2% dextran is added to each tube, mixed and spinned briefly. Approximately 5 minutes after adding Cab-osil M-5, the tubes are centrifuged for 5 minutes, 14000 rpm (minifuge). 200 μl of the supernatant is transferred to a microscint-tube and 3.5 ml atomlight is added. Tubes are measured in a liquid scintillation counter. If ³H FF-MAS binding can be displaced by unlabelled FF-MAS, then the protein tested is a MAS receptor or a MAS signalling protein.

### EXAMPLE 4

### Assay to Determine Whether a Specific Compound is a Ligand

10 μl of the compound to be tested (1, 3, 10, 30, 100, 300, 1000, 3000 nM in 6.6% EtOH) is mixed with 10 μl of 200 nM ³H-labelled FF-MAS (approximately 12.8 Ci/mmol) in assay buffer (10 mM Tris; 1.5 mM EDTA; 10% glycerin; 1.0 mM CHAPS; 1% BSA). 10 μg of SAM1a protein freshly diluted in assay buffer, is added to give a final volume of 40 μl. Unspecific binding is measured in the presence of 30 μM unlabelled FF-MAS, total binding is determined by adding 10 μl assay buffer containing 6.6% EtOH. Incubation is performed for 2 hours at 4° C. 250 μl of ice-cold assay buffer containing 2% Cab-osil and 0.2% dextran is added to each tube, mixed and spinned briefly. Approximately 5 minutes after adding Cab-osil, the tubes are centrifuged for 5 minutes, 14000 rpm (minifuge). 200 μl of the supernatant is transferred to a microscint-tube and 3.5 ml atomlight is added. Tubes are measured in a liquid scintillation counter. If a compound can displace specific FF-MAS binding, then it is a ligand to the MAS receptor or to a MAS signalling protein.

### EXAMPLE 5

### Cloning of SAM1

An cDNA library was prepared from mRNA isolated from 10,000 oocytes, from 24 days old mice. The cDNA library was constructed in the pSPORT plasmid vector (LifeTechnologies). Clones were picked at random and partially sequenced, and the sequences were assembled using phred/phrap programs. Out of several thousand clones that were sequenced, an assembly of two exhibited 21% amino acids identity to a human Oxysterol Binding Protein. The longest clone MOCY2864 was completely sequenced and no identical or orthologous genes were found in the databases. This new gene was named SAM1. Amplification of the 5′ end of SAM1cDNA was performed by PCR on the oocyte library using a primer specific for pSPORT, #176959 (SEQ ID NO: 9) and a primer specific for SAM1 #198241 (SEQ ID NO: 10). This revealed cDNAs with two different 5′ ends, which were designated SAM1a and SAM1b. Full-length PCR amplification was done on the mouse oocyte library using primer #199772 (SEQ ID NO: 11) and #198239 (SEQ ID NO: 12) for SAM1a and #201790 (SEQ ID NO: 13) and #198239 (SEQ ID NO: 12) for SAM1b. Recognition sites for NheI and NotI, respectively, were incorporated in the primers. SAM1a and SAM1b cDNAs were digested with NheI and NotI restriction enzymes and cloned into pcDNA3,1+(Invitrogen).

DNA constructs directing the expression of SAM1a and SAM1b proteins fused to a C-terminal histidine stretch, which could be used for in purification because of its affinity to nickel-columns were made as follows. SAM1a and SAM1b cDNAs were PCR amplified using the primers #199772 (SEQ ID NO: 11) and #211465 (SEQ ID NO: 14) and primers #201790 (SEQ ID NO: 13) and #211465 (SEQ ID NO: 14), respectively. Recognition sites for NheI and XmaI, respectively, were incorporated in the primers. The PCR-products were then cloned into pBlueBac4,5V5HIS (Invitrogen) using the restriction enzymes NheI and XmaI. This intermediate construct was then digested by SmaI and BstBI, filled-in using the Klenow fragment of DNA polymerase1 and then religated. These construct were designated “SAM1a in pBlueBac4,5V5HIS” and “SAM1b in pBlueBac4,5V5HIS”.

SAM1Expression in Sf9 Cells

SAM1a-HIS and SAM1b-HIS proteins were expressed using recombinant Baculo virus in Sf9 cells according to the “Bac-N-Blue™ Transfection Kit” manual (Invitrogen).

To infect Sf9 insect cells 0,5 μg Bac-N-Blue™ DNA and the recombinant transfer plasmid “SAM1 in pBlueBac4,5V5HIS” (4 μg) were incubated with 1 ml Grace's Insect Media and and 20 μl Insectin-Plus Liposomes for 15 minutes, then the mixture was added to 2×10⁶ Sf9 cells in a 60 mm dish. The cells were left for 96 hours rocking at 27° C. Vira were isolated and plaque assay was performed. Putative recombinant plaques were picked and P-1 viral stocks were made. PCR analysis of recombinant viral clones was done and from positive clones high-titer viral stocks were then prepared. For large-scale SAM1a and SAM1b expression 500 ml Sf9 cells (2,0×10⁶ cells/ml) was infected with 25 ml virus (1,8×108 plaque forming units/ml) or 60 ml virus (6×10⁷ pfu/ml) for SAM1a and SAM1b, respectively. After 70 hours of incubation the cells were pelleted by centrifugation and the protein was purified.

SEQ ID NO: 1 and SEQ ID NO: 3 are the nucleotides of the cDNA from two mouse MAS receptors or signalling peptides, designated SAM1a and SAM1b, respectively, and having the amino acid sequences stated in SEQ ID NO: 2 and SEQ ID NO: 4, respectively. SEQ ID NO: 5 and SEQ ID NO: 7 are the nucleotides of the cDNA from two human MAS receptors or signalling peptides, designated SAM1a and SAM1b, respectively, and having the amino acid sequences stated in SEQ ID NO: 6 and SEQ ID NO: 8.

### EXAMPLE 6

### Purification of SAM1a and SAM1b

Purification of HIS-SAM1a and HIS-SAM1b was performed according to the manual of QIAGEN: The QIAexpressionist forth edition.

Briefly, cell cultures of SF9 insect cells (containing the construct for 6×His-SAM1a or 6×His-SAM1b in a baculovirus expression vector) were centrifuged, pellets were lysed by addition of lysis buffer (50 mM NaH₂PO₄, 300 mM NaCl, 10 mM imidazole, pH 8) and lysozyme, sonication on ice 6×10 seconds, where after the lysates were cleared by centrifugation.

Cleared lysates were incubated under mild agitation with 50% slurry of Ni-NTA agarose (QIAGEN), for binding of 6×His-SAM1a, washed twice (50 mM NaH₂PO₄, 300 mM NaCl, 20 mM imidazole, pH 8) and eluted with high concentration of imidazole (50 mM NaH₂PO₄, 300 mM NaCl, 250 mM imidazole, pH 8). Purified proteins were analysed on Coomassie (Brilliant Blue, Sigma) stained SDS-polyacrylamide gels (NuPAGE 4-12% Bis-Tris gel, Invitrogen) for evaluation of yield and purity.

What is claimed is:

1. An isolated and/or purified polynucleotide which hybridises at high stringency to an oligonucleotide of 25 or more contiguous nucleotides of SEQ ID NO: 1 or SEQ ID NO: 3 and which polynucleotide codes for a) a MAS receptor or for a MAS signalling protein; or b) a ligand binding domain of a MAS receptor or MAS signalling protein.

2. The isolated polynucleotide of claim 1, wherein the MAS receptor or signalling protein has the ability to bind to 3β-hydroxy-4,4-dimethylcholest-8,14,24-triene (FF-MAS).

3. The isolated polynucleotide of claim 1, which is a RNA antisense sequence.

4. The isolated polynucleotide of claim 1, which is a cDNA sequence.

5. The isolated polynucleotide of claim 1, which encodes a polypeptide displaying MAS receptor or MAS signalling protein activity.

7. The isolted polynucleotide of claim 1, which encodes a MAS receptor or MAS signalling protein comprising the amino acid sequence of SEQ ID NO: 2 or SEQ ID NO: 4.

8. The isolated polynucleotide of claim 1, comprising the sequence of SEQ ID NO: 1 or SEQ ID NO: 3.

9. A probe of at least 12 nucleotides, said probe being capable of hybridising with nucleic acids which encode a MAS receptor or MAS signalling protein.

10. The probe of claim 9, comprising an oligonucleotide or polynucleotide of 25 or more contiguous nucleotides of SEQ ID NO: 1 or SEQ ID NO: 3 capable of specifically hybridising with a gene which encodes a MAS receptor or MAS signalling protein, or allelic and species variants thereof.

11. The probe of claim 9, comprises from about 40 to about 60 nucleotides in length.

12. The probe of claim 9, which is labelled to provide a detectable signal.

13. The probe of claim 9, comprising the nucleotides of SEQ ID NO: 1 or SEQ ID NO: 3.

14. A DNA construct comprising a DNA sequence which hybridises at high stringency to an oligonucleotide or polynucleotide of 25 or more contiguous nucleotides of SEQ ID NO: 1 or SEQ ID NO: 3 and which encodes a) a MAS receptor or MAS signalling protein; or b) a ligand binding domain of a MAS receptor or MAS signalling protein.

15. The DNA construct of claim 14, wherein the DNA sequence encodes a MAS receptor or MAS signalling protein having the amino acid sequence of SEQ ID NO: 2 or SEQ ID NO: 4.

16. A cultured cell line, yeast or bacteria transformed or transfected with a DNA construct which comprises a DNA sequence which hybridises at high stringency to an oligonucleotide or polynucleotide of 25 or more contiguous nucleotides of SEQ ID NO: 1 or SEQ ID NO: 3 and which encodes a) a MAS receptor or MAS signalling protein; or b) a ligand binding domain or a transmembrane domain of a MAS receptor or MAS signalling protein.

17. The cell line, yeast or bacteria of claim 16, which does not express endogenous MAS receptor or MAS signalling proteins.

18. An isolated and/or purified MAS receptor or MAS signalling protein, or peptide fragment or salt thereof.

19. The isolated and/or purified protein of claim 18, comprising the amino acid sequence of SEQ ID NO: 2 or SEQ ID NO: 4, or a fragment of said amino acid sequence able to bind to FF-MAS.

20. An isolated antibody which specifically binds to a MAS receptor or MAS signalling protein.

21. The isolated antibody of claim 20, wherein said antibody is a monoclonal antibody.

22. The isolated antibody of claim 20, which blocks the binding of MAS to a MAS receptor or MAS signalling protein.

23. A hybridoma which produces the monoclonal antibody of claim 21.

24. A method for detecting the presence of a compound or a salt thereof which has affinity for a MAS receptor or MAS signalling protein, comprising the steps of a) contacting the compound with the MAS receptor or MAS signalling protein, a peptide fragment thereof or a salt thereof; and b) measuring the affinity of said compound for the MAS receptor or MAS signalling protein.

25. The method of claim 24, comprising the steps of a) exposing a compound in the presence of a MAS agonist including MAS to a MAS receptor or MAS signalling protein coupled to a response pathway under conditions and for a time sufficient to allow binding of the compound to the MAS receptor or MAS signalling protein and an associated response through the pathway; and b) detecting a reduction in the stimulation of the response pathway resulting from the binding of the compound to the MAS receptor or MAS signalling protein, relative to the stimulation of the response pathway by the MAS agonist alone and there from determining the presence of a MAS antagonist.

26. The method of claim 24, comprising the steps of a) exposing a compound in the presence of a MAS antagonist to a MAS receptor or MAS signalling protein coupled to a response pathway under conditions and for a time sufficient to allow binding of the compound to the MAS receptor or MAS signalling protein and an associated response through the pathway; and b) detecting an increase of the stimulation of the response pathway resulting from the binding of the compound to the MAS receptor or MAS signalling protein, relative to the stimulation of the response pathway by the MAS antagonist alone and there from determining the presence of a MAS agonist.

27. A compound or a salt thereof which has affinity for the MAS receptor or a MAS signalling peptide, detected by the method of claim 24.

28. A method for producing a MAS receptor or MAS signalling protein having the amino acid sequence of SEQ ID NO: 2 or SEQ ID NO: 4, which comprises a) growing cells, yeast or bacteria transformed or transfected with a DNA construct which comprises a DNA sequence of SEQ ID NO: 1 or SEQ ID NO: 3 coding for the expression of the MAS receptor or MAS signalling protein, and b) isolating the MAS receptor or MAS signalling protein from the cells.

29. The method of claim 28, wherein the MAS receptor or MAS signalling protein is isolated by immunoaffinity purification.

30. A kit for screening a compound or a salt thereof which has affinity for a MAS receptor or MAS signalling protein, which contains the MAS receptor or MAS signalling protein, the peptide fragment thereof or a salt thereof.

31. A MAS receptor, which comprises the amino acid sequence shown in SEQ ID NO: 2, or an analogue thereof binding FF-MAS, with an affinity constant below 100 μM, preferably below 10 μM.

32. The MAS receptor of claim 31, with the proviso that it is different from the amino acid sequence in SEQ ID NO: 6 and 8.

33. A DNA sequence encoding the MAS receptor of claim 32.

34. A MAS receptor, which comprises the amino acid sequence shown in SEQ ID NO: 4, or an analogue thereof binding FF-MAS, with an affinity constant below 100 μM, preferably below 10 μM.

35. The MAS receptor of claim 34, with the proviso that it is different from the amino acid sequence in SEQ ID NO: 6 and 8.

36. A DNA sequence encoding the MAS receptor of claim 35.

37. A MAS receptor, which comprises the partial amino acid sequence shown in SEQ ID NO: 6, or an analogue thereof binding FF-MAS, with an affinity constant below 100 μM, preferably below 10 μM.

38. The MAS receptor of claim 35, with the proviso that it is different from the amino acid sequence in SEQ ID NO: 6 and 8.

39. A MAS receptor, which comprises the partial amino acid sequence shown in SEQ ID NO: 8, or an analogue thereof binding FF-MAS, with an affinity constant below 100 μM, preferably below 10 μM.

40. The MAS receptor of claim 37, with the proviso that it is different from the amino acid sequence in SEQ ID NO: 6 and 8.

41. A DNA construct which comprises a DNA sequence encoding a MAS receptor or a functional analog thereof, with the proviso that it is different from the nucleotides of SEQ ID NO: 5 and 7.

42. A DNA construct comprising the DNA sequence shown in SEQ ID NO: 1, or a fragment thereof, or a DNA sequence encoding a functional analogue thereof binding FF-MAS, with an affinity constant below 100 μM, preferably below 10 μM.

43. The DNA construct of claim 42, with the proviso that it is different from the nucleotides of SEQ ID NO: 5 and 7.

44. A DNA construct comprising a DNA sequence shown in SEQ ID NO: 5, or a DNA sequence coding for a functional analogue thereof binding FF-MAS, with an affinity constant below 100 μM, preferably below 10 μM.

45. The DNA construct of claim 44, with the proviso that it is different from the nucleotides of SEQ ID NO: 5 and 7.

46. A method of screening for ligands to a MAS receptor, the method comprising incubating a MAS receptor with a substance suspected to be an agonist or antagonist of FF-MAS, and subsequently with FF-MAS, or an analogue thereof, and detecting any effect of binding of FF-MAS, or the analogue to the MAS receptor.

47. A method of screening for ligands to the MAS receptor, the method comprising incubating FF-MAS, or an analogue thereof with a substance suspected to be an agonist or antagonist of activity of FF-MAS, and subsequently with a MAS receptor according to any one of the preceding claims to a MAS receptor, and detecting any effect of binding of FF-MAS, or the analogue to the receptor.
